(19) United States
(12) Patent Application Publication
(10) Pub. No.: US 2021/0282412 A1
(43) Pub. Date: Sep. 16, 2021

(54) FROZEN FRESH YEAST FORMED BODY AND METHOD FOR PRODUCING SAME

(71) Applicant: KANEKA CORPORATION, Osaka-shi (JP)

(72) Inventors: Dai FUKUDA, Takasago-shi (JP); Takuya IZUMI, Takasago-shi (JP)

(73) Assignee: KANEKA CORPORATION, Osaka-shi (JP)

(21) Appl. No.: 16/327,026

(22) PCT Filed: Aug. 25, 2017
(86) PCT No.: PCT/JP2017/030471

§ 371 Date: Feb. 21, 2019

(30) Foreign Application Priority Data
Aug. 26, 2016 (JP) 2016-166205

(51) Int. Cl. A21D 8/04; C12N 1/18

(52) U.S. Cl. CPC A21D 8/047; C12N 1/185; C12N 1/18

(57) ABSTRACT

Disclosed is a frozen fresh yeast formed body that is obtained by freezing fresh yeast without adding edible fat and oil or an emulsifier, maintains the leavening power of fresh yeast even after long-term storage, is not tightly consolidated with other frozen formed bodies and can be easily separated from other frozen formed bodies even when consolidated with the other frozen formed bodies, and is countable to weigh the amount of yeast. The frozen fresh yeast formed body is obtained by freezing fresh yeast and has an almost spherical shape whose maximum width is 2 to 20 cm. The frozen fresh yeast formed body has a yeast content of 25 to 40% by weight in terms of dry weight and a moisture content of 60 to 75% by weight with respect to a total weight thereof. It is preferred that a maximum contact area between the frozen fresh yeast formed bodies is 20% or less of a total surface area of the single formed body.

### TECHNICAL FIELD

The present invention relates to a frozen fresh yeast formed body obtained by freezing fresh yeast and a method for producing the same.

### BACKGROUND ART

In the bread industry, breadmaking processes are required to be simpler and more efficient because inexperienced workers have recently increased due to the mobilization of human resources and internationalization, and cost competition has recently increased.

The distribution form of baker's yeast is roughly divided into fresh yeast, dry yeast, and semi-dry yeast. Fresh yeast is also called compressed yeast, and is sold in the form of a block or powder. Such fresh yeast is most widely distributed because its features are supported by the market, such as higher ability to decompose sugar, better flavor, and higher leavening power than dry yeast or semi-dry yeast.

However, fresh yeast is vulnerable to dryness and sensitive to a change in temperature, and therefore needs to be stored under refrigeration. If not stored under refrigeration, fresh yeast quickly goes bad. Even when fresh yeast is stored under refrigeration, its storage life is generally said to be two weeks, and the longest being one month.

In addition, fresh yeast is fragile and is therefore easily disintegrated during production, distribution, storage, and weighing operations, which makes weighing operations before blending with bread dough complicated. Further, there are problems associated with weighing operations such as a hygiene problem and a problem that cleaning needs to be performed. Such problems become obstacles to simplification and efficiency improvement in breadmaking processes.

On the other hand, in order to extend the storage life of fresh yeast, fresh yeast may be frozen for storage. However, Non-Patent Literature 1 discloses that it is better not to freeze fresh yeast because when fresh yeast is frozen for storage, its leavening power is weakened due to the death of some of yeast cells.

Patent Literature 1 discloses granular frozen yeast. However, this granular frozen yeast is one obtained simply by freezing dry or semi-dry yeast having a dry matter content of 70 to 85%, and therefore has a problem that, unlike the above-described fresh yeast, its ability to decompose sugar is poor and its leavening power is low.

Patent Literature 2 discloses frozen fresh yeast in the form of pellets, and also disclose that this frozen fresh yeast is obtained by freezing fresh yeast so that long-term storage of fresh yeast is achieved. The frozen fresh yeast pellets disclosed in Patent Literature 2 are pellets obtained by kneading compressed fresh yeast with edible fat and oil or an emulsifier or pellets whose surfaces are coated with edible fat and oil or an emulsifier, and 40% or less of the pellets pass through a sieve whose lattice width is 0.9 mm, and 80% or more of the pellets pass through a sieve whose lattice width is 25 mm Patent Literature 2 discloses that the frozen fresh yeast pellets are excellent in measurability and handleability during its use and dispersibility in wheat dough, and can be used in the same way as conventional fresh yeast.

### CITATION LIST

### Patent Literature

PTL 1: JP-A-62-282578
PTL 2: JP-A-H09-84579

### Non-Patent Literature

Non-PTL 1: Journal of Biochemistry, 1997, vol. 54, pp. 234-240

### SUMMARY OF INVENTION

### Technical Problem

However, in order to produce the frozen fresh yeast pellets disclosed in Patent Literature 2, an extra raw material, such as edible fat and oil or an emulsifier, needs to be added. Further, the specifically-disclosed frozen fresh yeast pellets have a particle diameter as small as 3 mm (Example), and therefore there is a problem that it is difficult to count the number of the pellets to weigh the amount of yeast.

Further, a conventional formed body obtained by freezing fresh yeast without using edible fat and oil or an emulsifier has a problem that particularly when unfrozen formed bodies that are in close contact with each other are frozen, the frozen formed bodies are tightly consolidated together and therefore cannot be easily separated from each other, which makes it difficult to count the number of the formed bodies to weigh the amount of yeast.

It is therefore an object of the present invention to provide a frozen fresh yeast formed body that is obtained by freezing fresh yeast without adding edible fat and oil or an emulsifier, maintains the leavening power of fresh yeast even after long-term storage, is not tightly consolidated with other frozen formed bodies and can be easily separated from other frozen formed bodies even when consolidated with the other frozen formed bodies, and is countable to weigh the amount of yeast.

### Solution to Problem

In order to achieve the above object, the present inventors have intensively studied and found that a frozen fresh yeast formed body made of fresh yeast, having an almost spherical shape whose maximum width is within a specific range, and having a yeast content and a moisture content within their respective specific ranges does not contain edible fat and oil or an emulsifier, has excellent long-term storage stability, is not tightly consolidated with other frozen formed bodies and can be easily separated from other frozen formed bodies even when consolidated with the other frozen formed bodies, and is countable to weigh the amount of yeast. This finding has led to the completion of the present invention.

More specifically, a first aspect of the present invention relates to a frozen fresh yeast formed body obtained by freezing fresh yeast, having an almost spherical shape having a maximum width of 2 to 20 cm, and having a yeast content of 25 to 40% by weight in terms of dry weight and a moisture content of 60 to 75% by weight with respect to a total weight thereof. It is preferred that a maximum contact area between the frozen fresh yeast formed bodies is 20% or less of a total surface area of the single formed body. It is preferred that the frozen fresh yeast formed body is obtained by freezing fresh yeast having a density of 0.90 to 1.20 g/cm².

A second aspect of the present invention relates to a package including a plurality of the frozen fresh yeast formed bodies being packed. It is preferred that a coefficient of variation for formed body weight is 0.050 or less.

A third aspect of the present invention relates to a method for producing a frozen fresh yeast formed body, including the step of forming fresh yeast into an almost spherical shape having a maximum width of 2 to 20 cm to obtain a formed body and freezing the formed body to obtain a frozen fresh yeast formed body, wherein the frozen fresh yeast formed body has a yeast content of 25 to 40% by weight in terms of dry weight and a moisture content of 60 to 75% by weight with respect to a total weight thereof.

A fourth aspect of the present invention relates to a method for producing bread dough, including the step of kneading the frozen fresh yeast formed body and other bread dough raw materials to produce bread dough. It is preferred that the frozen fresh yeast formed body is added to and kneaded with the bread dough raw material in a frozen state without being thawed or after being thawed.

A fifth aspect of the present invention relates to a method for making bread, including the step of cooking bread dough obtained by the above-described production method to obtain bread.

A sixth aspect of the present invention relates to a method for preventing consolidation of frozen fresh yeast formed bodies, including the step of forming fresh yeast into an almost spherical shape having a maximum width of 2 to 20 cm to obtain a formed body and freezing the formed body to obtain a frozen fresh yeast formed body, wherein the frozen fresh yeast formed body has a yeast content of 25 to 40% by weight in terms of dry weight and a moisture content of 60 to 75% by weight with respect to a total weight thereof.

### Advantageous Effects of Invention

According to the present invention, it is possible to provide a frozen fresh yeast formed body that is obtained by freezing fresh yeast without adding edible fat and oil or an emulsifier and that maintains the leavening power of fresh yeast even after long-term storage. Further, even when produced by freezing unfrozen formed bodies that are in close contact with each other, frozen fresh yeast formed bodies are not tightly consolidated together, and can be easily separated from each other even when consolidated together. Further, the number of the frozen formed bodies can be counted to weigh the amount of yeast, which makes it possible to weigh the amount of yeast more simply and efficiently.

### DESCRIPTION OF EMBODIMENTS

Hereinafter, the present invention will be described in more detail.

A frozen fresh yeast formed body (hereinafter also referred to as “frozen formed body”) according to the present invention is frozen fresh yeast formed into a predetermined shape. The frozen formed body is preferably one not containing edible fat and oil or an emulsifier, that is, one made of only fresh yeast and containing substantially no additive.

The frozen fresh yeast formed body according to the present invention is frozen, and therefore can be stored for a longer period of time as compared with unfrozen fresh yeast merely stored under refrigeration. Further, the frozen fresh yeast formed body maintains the leavening power of unfrozen fresh yeast even after long-term storage. Further, the frozen fresh yeast formed body according to the present invention is frozen, and is therefore less likely to lose its shape as compared with an unfrozen formed body even when subjected to external impact during production, distribution, storage, and weighing operations. Therefore, the formed bodies are uniform in weight, which is advantageous in that the amount of yeast can be weighed simply by counting the number of the formed bodies.

The fresh yeast used in the present invention is yeast also called compressed yeast and having a high moisture content. The fresh yeast is different in this point from dry yeast (moisture content: 5 to 10% by weight) or semi-dry yeast (moisture content: 15 to 30% by weight) that is subjected to a drying process to reduce a moisture content. The fresh yeast used in the present invention has a yeast content of 25 to 40% by weight in terms of dry weight and a moisture content of 60 to 75% by weight when the total weight of the frozen fresh yeast formed body is taken as 100%. If the moisture content is less than 60% by weight, there is a case where the yeast is difficult to maintain a predetermined shape, which makes it difficult to form the yeast into a predetermined shape. On the other hand, if the moisture content is larger than 75% by weight, the yeast is sticky, and therefore there is a fear that the formed bodies are likely to be consolidated together, and further the leavening power of the fresh yeast is reduced after long-term storage, and the formed body tends to be easily disintegrated. The yeast content is preferably 30 to 35% by weight, and the moisture content is preferably 65 to 70% by weight.

The strain of the yeast used in the present invention is not particularly limited as long as its leavening power is not significantly reduced by frozen storage, and examples thereof include the following Saccharomyces cerevisiae strains: CFB27-1 (accession number: FERM BP-15903, described in Japanese Patent No. 4357007, used in Examples described later and shown in Table 1), KCY1160 (accession number: FERM P-16962, described in Japanese Patent No. 4475144), KCY 1170 (accession number: FERM P-20408, described in Japanese Patent No. 4475144), KSY290 (accession number: FERM P-18863, described in Japanese Patent Nos. 4411864 and 4513383), KSY68-9290 (accession number: FERM P-20204, described in Japanese Patent No. 4839809), KSY85-596 (accession number: FERM P-20295, described in Japanese Patent No. 4839809), KKK47 (accession number; FERM BP-7267, described in Japanese Patent No. 4565789), KGLY59 (accession number: FERM BP-20635, described in Japanese Patent No. 4839860, used in Examples described later and shown in Table 8), KCY1254 (accession number: NITE BP-1396, described in Japanese Patent No. 5677624, used in Examples described later and shown in Table 9), KCY1240 (accession number: NITE BP-1269, described in Japanese Patent No. 5677624), KCY1249 (accession number: NITE BP-1270, described in Japanese Patent No. 5677624), KCY1251 (accession number: NITE BP-1272, described in Japanese Patent No. 5677624), KCY1217 (accession number: NITE BP-1058, described in Japanese Patent No. 5907161, used in Examples described later and shown in Table 10), KCY1222 (accession number: NITE BP-1059, described in Japanese Patent No. 5907161, used in Examples described later and shown in Table 11), KSY735 (accession number: NITE P-731, described in Japanese Patent No. 5926494), KSY736 (accession number: NITE P-1071, described in Japanese Patent No. 5926494), and KSY737 (accession number: NITE P-1072, described in Japanese Patent No. 5926494).

The predetermined shape of the frozen fresh yeast formed body according to the present invention is almost spherical. The almost spherical shape means a rounded shape, and specific examples thereof include a spherical shape, an ellipsoidal shape, a cylindrical shape, and a barrel-like shape (rounded cylindrical shape). Among them, from the viewpoint of the effects of the present invention, the frozen fresh yeast formed body according to the present invention preferably has a shape not including a flat surface, and more preferably has a spherical shape or an ellipsoidal shape.

When the shape of the formed body is almost spherical, the surface area of the frozen formed body is reduced, that is, the area of contact between the frozen formed bodies is reduced. Therefore, the frozen formed bodies are less likely to be consolidated together, and the frozen formed bodies are less likely to form a tightly consolidated mass even when consolidated together. Therefore, even when the formed bodies are consolidated together to form a consolidated mass, the consolidated mass can be easily divided with a standard grip strength of general adult of about 5 kg/cm², which makes it possible to count the number of the formed bodies to easily weigh the amount of yeast. On the other hand, if the frozen formed body has an angular shape such as a rectangular parallelepiped shape or a cubic shape, the area of contact between the frozen formed bodies is large, and therefore the frozen formed bodies are likely to be consolidated together or to form a tightly consolidated mass that cannot be easily divided. Therefore, there is a case where it is not easy to count the number of the formed bodies to weigh the amount of yeast.

From the above viewpoint, the ratio of the maximum area of contact between the formed bodies to the total surface area of the single frozen fresh yeast formed body (hereinafter referred to as a maximum contact area ratio) is preferably small. If the maximum contact area ratio is too large, there is a fear that the formed bodies are tightly consolidated together to form a consolidated mass. In this case, the consolidated mass is difficult to divide, and therefore there is a fear that the number of the formed bodies cannot be easily counted to weigh the amount of yeast. More specifically, the maximum contact area ratio is preferably 20% or less, more preferably 16% or less, even more preferably 10% or less. It is to be noted that the maximum contact area ratio is a value calculated on the basis of the shape of the frozen formed body. For example, when the formed body is spherical or ellipsoidal, the maximum contact area ratio is 0%, and when the formed body is cylindrical, the maximum contact area ratio is the ratio of the surface area of both the bottom surfaces to the total surface area of the cylinder.

The frozen fresh yeast formed body according to the present invention has an almost spherical shape whose maximum width is 2 cm or more but 20 cm or less. When the maximum width is smaller than 2 cm, the formed body is too small, which makes it difficult to count the number of the formed bodies to weigh the amount of yeast. Further, the surface of the frozen formed body tends to be sticky, which makes it difficult to handle the frozen formed bodies to weigh the amount of yeast. Further, the frozen formed bodies are likely to be consolidated together to form a consolidated mass, and the consolidated mass is difficult to divide. Also from the viewpoint, it is difficult to count the number of the formed bodies to weigh the amount of yeast. On the other hand, if the maximum width is larger than 20 cm, the frozen formed body is difficult to hold by hand, and a consolidated mass of the frozen formed bodies cannot be easily divided, which makes it difficult to count the number of the formed bodies to weigh the amount of yeast. The maximum width is preferably 3 cm or more but 15 cm or less, more preferably 4 cm or more but 10 cm or less. It is to be noted that the maximum width means the maximum diameter or side length of the almost spherical shape. In the case of a spherical shape, the maximum width refers to the diameter, in the case of an ellipsoidal shape, the maximum width refers to the largest diameter among three diameters, and in the case of a cylindrical shape, the maximum width refers to the height or the diameter of the bottom surface, whichever the larger.

When the frozen fresh yeast formed body according to the present invention has a shape other than a spherical shape, such as an ellipsoidal shape or a cylindrical shape, the minimum width thereof is not particularly limited. However, if the minimum width is too small, the surface of the frozen formed body tends to be sticky, and therefore there is a fear that the frozen formed bodies are likely to be consolidated together, and the formed body is likely to lose its shape. Therefore, the minimum width is preferably 0.5 cm or more, more preferably 1 cm or more, even more preferably 2 cm or more.

The weight of the single frozen fresh yeast formed body according to the present invention is not particularly limited. However, if the weight of the single formed body is too small, the formed bodies are likely to be consolidated together to form a large consolidated mass, which makes it difficult to count the number of the formed bodies to weigh the amount of yeast. Further, the surface of the frozen formed body is easily melted and becomes sticky, and therefore there is a fear that the frozen formed bodies are difficult to handle to count the number of the frozen formed bodies, and the coefficient of variation for formed body weight increases. On the other hand, if the weight of the single formed body is too large, it takes time to freeze the formed body, and the formed body is likely to lose its shape during production and distribution. As a result, there is a fear that the coefficient of variation for formed body weight increases. From the above viewpoint, the weight of the single frozen formed body is preferably 1 to 1000 g, more preferably 3 to 500 g, even more preferably 10 to 300 g.

The density of the frozen fresh yeast formed body according to the present invention is not particularly limited. However, if the density of the fresh yeast just before freezing is too small, the formed body is likely to lose its shape when subjected to external impact, and as a result, there is a fear that the coefficient of variation for formed body weight increases. On the other hand, if the density of the fresh yeast just before freezing is too large, forming is difficult to perform. From the above viewpoint, the density of the fresh yeast is preferably adjusted to 0.90 to 1.20 g/cm³, more preferably 1.00 to 1.10 g/cm³ before freezing.

A method for producing the frozen fresh yeast formed body according to the present invention is not particularly limited. The frozen fresh yeast formed body may be produced by forming the fresh yeast into a predetermined shape by compression or cutting before freezing, or may be produced by freezing massive fresh yeast such as block-shaped fresh yeast and then forming the frozen massive fresh yeast into a predetermined shape by cutting. However, the former production method is preferred from the viewpoint that forming is easily performed and the formed body is less likely to disintegrate.

A specific forming method is not particularly limited, and examples thereof include a method in which the fresh yeast is placed in a mold having a predetermined shape and pressure is applied to the fresh yeast, and a method in which the fresh yeast is formed into a predetermined shape by extrusion. Further, the density of the formed body can be adjusted by the pressure during the forming. Further, the density of the formed body may be adjusted by adjusting the pressure applied during the forming. A specific method used therefor is not particularly limited. For example, the pressure applied during the forming may be adjusted depending on the density of the formed body measured using a 3D laser volume meter, Selnac-WinVM210 (manufactured by ASTEX) during the forming.

The freezing may be either quick freezing or slow freezing, and the cooling rate during freezing is not particularly limited.

A package can be provided in which a plurality of the frozen fresh yeast formed bodies according to the present invention are packed in one container. This package may be formed by putting the frozen formed bodies into one container or by freezing unfrozen fresh yeast formed bodies put into one container. The number of the frozen formed bodies packed in one container is not particularly limited, but is, for example, preferably about 2 to 1000, more preferably 5 to 500, even more preferably 10 to 100.

The container is not particularly limited as long as it can store the frozen formed bodies therein and can hold the frozen formed bodies under freezing conditions, and examples thereof include a box, a bag, a bottle, and a cup. Alternatively, the container may be formed from package paper. The material of the container is not particularly limited, but the container preferably has a resin layer formed on at least its inner surface because the frozen formed bodies are hard to adhere to the inner wall of the container. The opening of the container is preferably sealable.

When the frozen bodies are packed in such a package, in general, there is a fear that the frozen bodies come into contact with each other and are consolidated together. However, the frozen formed bodies according to the present invention are not tightly consolidated together even when coming into contact with each other, and can be easily separated from each other even when consolidated together. Therefore, the frozen formed bodies can be taken out of the package to count the number of the frozen formed bodies to weigh the amount of yeast.

Further, variations in weight among the frozen formed bodies packed in the package are preferably small. When variations in weight among the formed bodies are smaller, the amount of yeast can be more accurately weighed by counting the number of the formed bodies. More specifically, the coefficient of variation for formed body weight is preferably 0.050 or less. The coefficient of variation for formed body weight is more preferably 0.014 or less, more preferably 0.010 or less, even more preferably 0.003 or less.

The coefficient of variation for formed body weight is an index indicating variations in weight among the formed bodies. When the coefficient of variation is smaller, variations in weight among the formed bodies are smaller. The coefficient of variation is calculated by, for example, randomly selecting 40 formed bodies, measuring the weight of each of the 40 formed bodies, calculating the average and standard deviation of weights of the formed bodies on the basis of the measurement results, and dividing the standard deviation by the average. When the number of the frozen formed bodies contained in one package is less than 40, the coefficient of variation may be calculated for 40 frozen formed bodies randomly collected from a plurality of packages. When a consolidated mass of the formed bodies is formed in the package, the weight of the single formed body is measured after the consolidated mass is divided into the individual formed bodies. Even when the frozen formed bodies according to the present invention are consolidated together to form a consolidated mass, the consolidated mass can be easily divided into the individual frozen formed bodies. Therefore, such a low coefficient of variation as described above can be achieved even when the coefficient of variation is calculated from the weights of the frozen formed bodies measured after the consolidated mass is divided into the individual frozen formed bodies.

When bread dough is produced using the frozen fresh yeast formed body according to the present invention, the same amount of the frozen fresh yeast formed body as yeast used in a conventional method is mixed and kneaded with other bread dough raw materials. Then, the mixture is subjected to primary fermentation, if necessary, and divided and shaped to obtain bread dough. The bread dough may be one subjected to secondary fermentation (final fermentation) after shaping or one before secondary fermentation. The bread dough may be frozen. If necessary, the other bread dough raw materials appropriately contains, in addition to cereal flour such as wheat flour, sugar, a dairy product, an egg(s), salt, an antioxidant, fat and oil, and water.

The frozen fresh yeast formed body according to the present invention may be thawed before added to and mixed with other bread dough raw materials, but may be added in a frozen state without being thawed. The frozen fresh yeast formed body according to the present invention is appropriately disintegrated even when frozen, and therefore can be disintegrated by stirring during mixing and dispersed in bread dough.

The bread dough is thawed and/or subjected to secondary fermentation, if necessary, and then can be cooked by a conventional method to make bread.

### EXAMPLES

The present invention will be more specifically described with reference to the following examples, but the present invention is not limited to these examples.

### Example 1

Compressed fresh yeast having a dry matter weight of 32% manufactured by KANEKA CORPORATION was placed in a mold having a shape and size shown in Table 1 and subjected to compression forming using a hand press machine to obtain a fresh yeast formed body having a shape, size, density, and weight (weight per one formed body) shown in Table 1. The dry matter weight of yeast in the formed body was 32.1%. In this example, CFB 27-1 (accession number: FERM BP-15903, described in Japanese Patent No. 4357007) was used as a yeast strain.

Then, the thus obtained 40 fresh yeast formed bodies were packed in a polyethylene bag and frozen in an air-cooled freezer at −20° C. for 2 months in a packed state to obtain a package containing 40 frozen fresh yeast formed bodies. It is to be noted that when the yeast formed bodies were packed in the bag such that they were in close contact with each other in the package.

### Examples 2 to 6

Fresh yeast formed bodies and a package containing frozen fresh yeast formed bodies were obtained in the same manner as in Example 1 in accordance with Table 1.

### Comparative Examples 1 to 3

Fresh yeast formed bodies were obtained and packed in a bag in the same manner as in Example 1 in accordance with Table 1 except that they were not frozen.

### Comparative Example 4

Five hundred grams of bulky (amorphous powdery) compressed fresh yeast having a dry matter weight of 32% manufactured by KANEKA CORPORATION was packed in a polyethylene bag and frozen in an air-cooled freezer at −20° C. for 1 week in a packed state to obtain a package containing bulky frozen fresh yeast. At this time, the weight of each bulk piece was in the range of 0.0001 to 5.0 g.

### Comparative Example 5

Five hundred grams of bulky compressed fresh yeast having a dry matter weight of 32% manufactured by KANEKA CORPORATION was granulated by a spherical granulator to obtain spherical fresh yeast formed bodies having a dimeter of 3.0 mm.

Then, all the fresh yeast formed bodies were packed in a polyethylene bag and frozen in an air-cooled freezer at −20° C. for 1 week in a packed state to obtain a package containing frozen fresh yeast formed bodies.

### Comparative Examples 6 and 7

Rectangular parallelepiped fresh yeast formed bodies and a package containing frozen fresh yeast formed bodies were obtained in the same manner as in Example 1 in accordance with Table 1.

### Reference Example 1

A package containing fine frozen fresh yeast obtained by kneading fresh yeast, edible fat and oil, and an emulsifier was obtained in the same manner as in Example 11 in Patent Literature 2. More specifically, the package was obtained in the following manner.

Five hundred grams of bulky compressed fresh yeast having a dry matter weight of 32% manufactured by KANEKA CORPORATION was kneaded with an emulsifier (sorbitan monostearate) and edible fat and oil (rapeseed hardened oil) in accordance with a formulation shown in Table 1, and then the kneaded product was granulated by a spherical granulator to obtain spherical fresh yeast formed bodies having a diameter of 3.0 mm. Then, all the fresh yeast formed bodies were packed in a polyethylene bag and frozen in an air-cooled freezer at −20° C. for 1 week in a packed state to obtain a package containing frozen fresh yeast.

### Test Example 1

The formed bodies and the packages containing the formed bodies obtained in Examples, Comparative Examples, and Reference Example were evaluated according to the following methods.

(1) Long-Term Storage Stability

The long-term storage stability of each of Examples 1 to 6, Comparative Examples 4 to 7, and Reference Example 1 was evaluated in the following manner. The amount of carbon dioxide generated with the unfrozen fresh yeast formed body stored under refrigeration at 4° C. for 1 day and the amount of carbon dioxide generated with the frozen fresh yeast formed body stored by freezing at −20° C. for 2 months were measured at 30° C. for 2 hours according to a method for measuring carbon dioxide from high-sugar dough established by Japan Yeast Industry Association as a baker's yeast test method. Then, the ratio of the amount of carbon dioxide generated with the frozen product stored by freezing for 2 months to the amount of carbon dioxide generated with the unfrozen product stored under refrigeration for 1 day. It is to be noted that the frozen fresh yeast formed body was directly subjected to the test in a frozen state without being thawed.

On the other hand, the long-term storage stability of each of Comparative Examples 1 to 3 was evaluated by measuring the amount of carbon dioxide generated with the fresh yeast formed body stored under refrigeration at 4° C. for 1 day and the amount of carbon dioxide generated with the fresh yeast formed body stored under refrigeration at 4° C. for 2 months and calculating the ratio of the amount of carbon dioxide generated with the product stored under refrigeration for 2 months to the amount of carbon dioxide generated with the product stored under refrigeration for 1 day.

(Evaluation criteria) 1: The amount of generated carbon dioxide was less than 70%. 2: The amount of generated carbon dioxide was 70% or more but less than 80%. 3: The amount of generated carbon dioxide was 80% or more but less than 90%. 4: The amount of generated carbon dioxide was 90% or more but less than 95%. 5: The amount of generated carbon dioxide was 95% or more.

(2) Difficulty of Disintegration

Each of the frozen fresh yeast formed bodies of Examples 1 to 6, Comparative Examples 4 to 7, and Reference Example 1 or each of the fresh yeast formed bodies of Comparative Examples 1 to 3 was lightly sandwiched between the palms of the upper and lower hands and rolled in the palms of the hands 10 times by moving, in opposite directions, the palms of the hands kept in parallel. The difficulty of disintegrating the formed body was evaluated according to the following criteria.

(Evaluation criteria) 1: Very easy to disintegrate, 2: Easy to disintegrate, 3: Slightly difficult to disintegrate, 4: Difficult to disintegrate, 5: Very difficult to disintegrate

(3) Difficulty of Consolidating Frozen Formed Bodies

The frozen formed bodies stored by freezing at −20° C. for 2 months after packaging were taken out of the package to observe whether or not the frozen formed bodies were consolidated together. The difficulty of consolidating the frozen formed bodies was evaluated according to the following criteria.

(Evaluation criteria) 1: The formed bodies were consolidated together to form a large mass. 2: The formed bodies were consolidated together to form a slightly large mass. 3: The formed bodies were consolidated together to form a small mass. 4: The formed bodies were not so much consolidated together. 5: The formed bodies were hardly consolidated together.

(4) Ease of Dividing Consolidated Mass

After packaging and frozen storage at −20° C. for 2 months, a mass of two or more frozen formed bodies consolidated together was taken out, and adjacent frozen formed bodies in the consolidated mass were respectively held by the left and right hands, and a force of about 5 kg/cm² (standard grip strength of general adult) was exerted on a portion where the frozen formed bodies consolidated together. The ease of dividing the consolidated mass was evaluated by whether or not the consolidated mass was easily divided.

(Evaluation criteria) 1: Very difficult to divide, 2: Difficult to divide, 3: Slightly easy to divide, 4: Easy to divide, 5: Very easy to divide

(5) Ease of Counting

The ease of counting the number of the frozen fresh yeast formed bodies of each of Examples 1 to 6, Comparative Examples 4 to 7, and Reference Example 1 was evaluated by comparison with the ease of counting the number of the block-shaped unfrozen fresh yeast formed bodies of each of Comparative Examples 1 to 3 by comprehensively considering the size of each of the formed bodies, the difficulty of disintegrating the formed body, the difficulty of consolidating the frozen formed bodies, and the case of dividing a consolidated mass.

(Evaluation criteria) 1: The counting was much more difficult as compared with Comparative Examples 1 to 3. 2: The counting was more difficult as compared with Comparative Examples 1 to 3. 3: The ease of counting was the same as those of Comparative Examples 1 to 3. 4: The counting was easier as compared with Comparative Examples 1 to 3. 5: The counting was much easier as compared with Comparative Examples 1 to 3.

(6) Coefficient of Variation for Weight

The package stored by freezing at −20° C. for 2 months was allowed to free fall from a height of 20 cm three times to divide a large consolidated mass into small consolidated masses inside the package, and the small consolidated masses were divided by applying a force of about 5 kg/cm′ thereto from the outside of the package. Then, the formed bodies were one by one taken out the package, and the weight of each of the formed bodies was measured. The standard deviation of the weights of the formed bodies was divided by the average of the weights of the formed bodies to calculate the coefficient of variation for weight. In the case of Comparative Examples 1 to 3, the unfrozen fresh yeast formed bodies were one by one taken out of the package stored under refrigeration at 4° C. for 1 day, and the weight of each of the formed bodies was measured and the coefficient of variation for weight was calculated in the same manner as described above.

The thus obtained results are shown in Table 1.

TABLE 1
Forms and evaluation results of fresh yeast formed bodies

| | | | Example | Example | Example | Example | Example | Example | Comparative |
|---|---|---|---|---|---|---|---|---|---|
| | | | 1 | 2 | 3 | 4 | 5 | 6 | Example 1 |
| Form | Shape | | Spherical | Spherical | Spherical | Ellipsoidal | Ellipsoidal | Cylindrical | Spherical |
| | Size | Diameter (cm) | 4.0 | 6.0 | 12.0 | — | — | 4.0 | 6.0 |
| | | Height (cm) | — | — | — | 1.0 | 4.0 | 8.0 | — |
| | | Long side (cm) | — | — | — | 4.0 | 6.0 | — | — |
| | | Short side (cm) | — | — | 2.0 | 4.0 | — | — | — |
| | Maximum contact | | 0 | 0 | 0 | 0 | 0 | 10.0 | 0 |
| | area ratio (%) | | | | | | | | |
| | Density (g/cm³) | | 1.06 | 1.06 | 1.06 | 1.07 | 1.07 | 1.01 | 1.06 |
| | Weight per one | | 36 | 120 | 959 | 4.5 | 54 | 101 | 120 |
| | formed body (g) | | | | | | | | |
| Formulation | Yeast content * 1 (wt %) | | 32.1 | 32.1 | 32.1 | 32.1 | 32.1 | 32.1 | 32.1 |
| | Moisture content (wt %) | | 67.9 | 67.9 | 67.9 | 67.9 | 67.9 | 67.9 | 67.9 |
| | Emulsifier content (wt %) | | — | — | — | — | — | — | — |
| | Fat and oil content (wt %) | | — | — | — | — | — | — | — |
| | Presence or | | Frozen | Frozen | Frozen | Frozen | Frozen | Frozen | Unfrozen |
| | absence of freezing | | | | | | | | |
| | treatment after forming | | | | | | | | |
| | Long-term storage stability | | 5 | 5 | 5 | 5 | 5 | 5 | 1 |
| | Difficulty of disintegration | | 5 | 5 | 5 | 5 | 5 | 5 | 3 |
| | Difficulty of consolidating | | 5 | 5 | 4 | 3 | 5 | 3 | — |
| | frozen formed bodies | | | | | | | | |
| | Ease of dividing | | 5 | 5 | 4 | 4 | 5 | 4 | — |
| | consolidated mass | | | | | | | | |
| | Ease of counting | | 5 | 5 | 4 | 4 | 5 | 4 | 3 |
| | Coefficient of variation | | 0.002 | 0.002 | 0.012 | 0.002 | 0.002 | 0.008 | 0.003 |
| | for weight | | | | | | | | |
| | | | Comparative | Comparative | Comparative | Comparative | Comparative | Comparative | Reference |
| | | | Example 2 | Example 3 | Example 4 | Example 5 | Example 6 | Example 7 | Example 1 |
| Form | Shape | | Cylindrical | Rectangular | Bulky | Spherical | Rectangular | Rectangular | Spherical |
| | | | | parallelepiped | | | parallelepiped | parallelepiped | |
| | Size | Diameter (cm) | 4.0 | — | — | 0.30 | — | — | 0.30 |
| | | Height (cm) | 8.0 | 6.5 | — | — | 6.5 | 6.5 | — |
| | | Long side (cm) | — | 6.5 | — | — | 11.7 | 6.5 | — |
| | | Short side (cm) | — | 5.9 | — | — | 3.3 | 5.9 | — |

| | | | | | | | | |
|---|---|---|---|---|---|---|---|---|
| | Maximum contact | 10.0 | 16.1 | — | — | 28.1 | 16.1 | 0 |
| | area ratio (%) | | | | | | | |
| | Density (g/cm³) | 1.01 | 1.04 | — | 1.01 | 1.04 | 1.04 | 1.01 |
| | Weight per one | 101 | 259 | 0.0001-5.0 | 0.014 | 261 | 259 | 0.014 |
| | formed body (g) | | | | | | | |
| Formulation | Yeast content * 1 (wt %) | 32.1 | 32.1 | 32.1 | 32.1 | 32.1 | 32.1 | 32.0 |
| | Moisture content (wt %) | 67.9 | 67.9 | 67.9 | 67.9 | 67.9 | 67.9 | 57.7 |
| | Emulsifier content (wt %) | — | — | — | — | — | — | 0.3 |
| | Fat and oil content (wt %) | — | — | — | — | — | — | 10.0 |
| | Presence or | Unfrozen | Unfrozen | Frozen | Frozen | Frozen | Frozen | Frozen |
| | absence of freezing | | | | | | | |
| | treatment after forming | | | | | | | |
| | Long-term storage stability | 1 | 1 | 1 | 5 | 5 | 5 | 5 |
| | Difficulty of disintegration | 3 | 2 | — | 5 | 5 | 5 | 4 |
| | Difficulty of consolidating | — | — | 1 | 2 | 1 | 1 | 5 |
| | frozen formed bodies | | | | | | | |
| | Ease of dividing | — | — | 1 | 1 | 1 | 2 | 5 |
| | consolidated mass | | | | | | | |
| | Ease of counting | 3 | 3 | 1 | 2 | 3 | 3 | 2 |
| | Coefficient of variation | 0.003 | 0.003 | 1.730 | 0.024 | 0.140 | 0.100 | 0.020 |
| | for weight | | | | | | | |

* 1 in terms of dry matter weight

As shown in Table 1, in the case of Examples 1 to 6, the amount of generated gas was kept large even after long-term storage, the frozen formed body was hard to disintegrate, the frozen formed bodies were hard to consolidate together, the frozen formed bodies were easily separated from each other even when consolidated together, and the number of the formed bodies was easily counted. On the other hand, in the case of Comparative Examples 1 to 3 in which the formed bodies were not frozen, the amount of generated gas was significantly reduced after long-term storage, and the formed body was relatively easily disintegrated. In the case of Comparative Example 4 in which bulky formed bodies were obtained, long-term storage stability was poor, the frozen formed bodies were easily consolidated together to form a consolidated mass not easily divided, and the number of the frozen formed bodies was difficult to count. In the case of Comparative Example 5 in which the formed bodies having a small maximum width were obtained or Comparative Examples 6 and 7 in which the rectangular parallelepiped formed bodies were obtained, the frozen formed bodies were easily consolidated together to form a consolidated mass not easily divided, and the number of the frozen formed bodies was difficult to count. In the case of Reference Example 1 in which additives such as edible fat and oil and an emulsifier were added, the formed bodies were very small, and therefore the number of the frozen formed bodies was difficult to count.

### Test Example 2

The frozen fresh yeast formed body of Example 2 or the unfrozen fresh yeast formed body of Comparative Example 1 was used to make bread (one-loaf bread and pullman bread) in accordance with Formulation 1 and Condition 1 shown in Table 2. It is to be noted that the frozen fresh yeast formed body was stored in a freezer at −20° C. for 3 months and then directly mixed in a frozen state with other bread dough raw materials without being thawed. The unfrozen fresh yeast formed body was stored under refrigeration at 4° C. for 1 day and then directly mixed with other bread dough raw materials.

TABLE 2
Formulation 1

| Baker's % | Sponge mixing | Dough mixing |
|---|---|---|
| Strong flour | 70 | 30 |
| Superfine sugar | 0 | 6 |
| Salt | 0 | 2 |
| Shortening | 0 | 5 |
| Yeast | 2 | 0 |
| Yeast food | 0.1 | 0 |
| Emulsifier | 0.25 | 0 |
| Skimmed milk | 0 | 2 |
| Water | 41 | 27 |

Condition 1

| | Mixing | L3M3 |
|---|---|---|
| Sponge | Dough kneading temperature | 24° C. |
| Mixing | Fermentation time | 240 min |
| Dough | Mixing | L2M5 ↓ L2M5 |
| mixing | Kneading time | 27° C. |
| | Floor time | 25 min |
| | Dough division | One-loaf: 350 g |
| | | Pullman: 240 g × 6 |
| | Bench time | 25 min |
| | Molding | One-loaf or Pullman |
| | Secondary fermentation | 39° C., Humidity 86% |
| | | One-loaf: 55 min |
| | | Pullman: 22 mm below |
| | | the top of the mold |
| | Baking | 180° C. |
| | | One-loaf: 30 min |
| | | Pullman: 38 min |

* The symbol “↓” indicates addition of shortening.
* Mixing was performed with a 20 quart mixer.

The quality of the thus obtained bread was evaluated on the basis of the amount of gas generated from sponge dough, the amount of gas generated from dough during secondary fermentation, the specific volume of the bread, and the taste of the bread which will be described below.

The amount of gas generated from sponge dough and the amount of gas generated from dough during secondary fermentation were measured in the following manner. The total amount of gas generated from 20 g of dough after sponge mixing and the total amount of gas generated from 20 g of dough after bench time were measured using Fermogaph II (manufactured by ATTO CORPORATION), the total amounts of generated gas were multiplied by 2.5, and the calculated values were regarded as the amounts of gas generated from 50 g of dough.

The specific volume of the bread was calculated by measuring the volume of the one-loaf bread with a 3D laser volume meter, Selnac-WinVM210 (manufactured by ASTEX) and dividing the volume by the weight of the one-loaf bread.

The taste of the pullman bread was evaluated by a sensory test according to the following criteria.

(Sensory Evaluation Criteria of Bread Taste)

Good: The taste was good and comparable to that of pullman bread made using unfrozen fresh yeast.

Not so good: The taste was slightly inferior to that of pullman bread made using unfrozen fresh yeast.

Poor: The taste was inferior to that of pullman bread made using unfrozen fresh yeast.

The thus obtained results are shown in Table 3.

TABLE 3

| | | Example 2 | Comparative Example 1 |
|---|---|---|---|
| | | Frozen fresh yeast | Unfrozen fresh yeast |
| Shape of formed body | | Spherical | Spherical |
| Size of formed body | | Diameter 6 cm | Diameter 6 cm |
| Amount of gas generated from sponge dough | (mL/4 hr) | 370 | 376 |
| Amount of gas generated from dough during | (mL/60 min) | 160 | 165 |
| secondary fermentation | | | |
| Specific volume of bread (one-loaf) | (cm³/g) | 4.9 | 5.0 |
| Taste of bread (Pullman) | — | Good | Good |

As can be seen from Table 3, the frozen fresh yeast formed body is excellent in long-term storage stability of yeast because the amount of gas generated from sponge dough, the amount of gas generated from dough during secondary fermentation, the specific volume of the bread, and the taste of the bread were all comparable to those when the unfrozen fresh yeast formed body was used in spite of the fact that the frozen fresh yeast formed body was used after storage by freezing for 3 months.

### Test Example 3

The frozen fresh yeast formed body of Example 2 or the unfrozen fresh yeast formed body of Comparative Example 1 was used to obtain molded medium-sugar bread dough in accordance with Formulation 2 and Condition 2 shown in Table 4, and then the bread dough was frozen and thawed to make bread rolls. It is to be noted that the frozen fresh yeast formed body was stored in a freezer at −20° C. for 3 months and then directly mixed in a frozen state with other bread dough raw materials without being thawed.

TABLE 4
Formulation 2

| Baker's % | |
|---|---|
| Strong flour | 100 |
| Superfine sugar | 15 |
| Salt | 1.6 |
| Shortening | 12 |
| Yeast | 5.3 |
| Conditioner | 0.6 |
| Skimmed milk | 3 |
| Egg | 12 |
| Water | 47 |
| Condition 2 | |
| Mixing | L2M5H1 ↓ L2M5H1 |
| Kneading time | 20° C. |
| Floor time | 10 min |
| Dough division | Roll: 80 g |
| Bench time | 5 min |
| Molding | Roll |
| Freezing | at −30° C. for 2 hours then |
| | at −20° C. for 3 months |
| Thawing | 25° C. 1 hr |
| Secondary fermentation | 39° C., Humidity 86% |
| | 55 min |
| Baking | 205° C. |
| | 11 min |

* The symbol “↓” indicates addition of shortening.
* Mixing was performed with a 20 quart mixer.

The quality of the thus obtained bread was evaluated on the basis of the amount of gas generated from dough during secondary fermentation, the specific volume of the bread, and the taste of the bread in the same manner as described above.

The thus obtained results are shown in Table 5.

TABLE 5

| | | Example 2 | Comparative Example 1 |
|---|---|---|---|
| | | Frozen fresh yeast | Unfrozen fresh yeast |
| Shape of formed body | | Spherical | Spherical |
| Size of formed body | | Diameter 6 cm | Diameter 6 cm |
| Among of gas generated from dough during | (mL/60 min) | 100 | 95 |
| secondary fermentation | | | |
| Specific volume of bread (roll) | (cm3/g) | 4.7 | 4.5 |
| Taste of bread (roll) | | Good | Good |

As can be seen from Table 5, the frozen fresh yeast formed body is excellent in long-term storage stability of yeast because the amount of gas generated from dough during secondary fomentation, the specific volume of the bread, and the taste of the bread were all comparable to those when the unfrozen fresh yeast formed body was used in spite of the fact that the frozen fresh yeast formed body was used after storage by freezing for 3 months.

### Test Example 4

The frozen fresh yeast formed body of Example 2 was thawed at 25° C. for 8 hours. In this case, the frozen fresh yeast formed body is regarded as being subjected to one freezing and thawing cycle. Then, freezing at −20° C. for 16 hours and thawing at 25° C. for 8 hours were performed once to three times. In this case, the frozen fresh yeast formed body is regarded as being subjected to two to four freezing and thawing cycles. The frozen fresh yeast formed bodies subjected to one to four freezing and thawing cycles were evaluated for their leavening power after thawing, shape retention ability during freezing, and texture after thawing according to the following criteria. The unfrozen fresh yeast formed body of Comparative Example 1 was evaluated for its leavening power and texture.

The leavening power was evaluated by measuring the amount of carbon dioxide generated at 30° C. for 2 hours in accordance with a method for measuring carbon dioxide generated from high-sugar dough established by Japan Yeast Industry Association as a baker's yeast test method.

The shape retention ability of the formed body during freezing was evaluated on a scale of 1 to 3 by whether or not the yeast formed body retained the same shape as the unfrozen fresh yeast formed body during freezing.

(Evaluation Criteria of Shape Retention Ability of Formed Body During Freezing)

Good: The shape was the same as that of the unfrozen fresh yeast formed body.

Not so good: The shape was slightly changed from that of the unfrozen fresh yeast formed body.

Poor: The shape was significantly changed from that of the unfrozen fresh yeast formed body.

The texture of the formed body was evaluated on a scale of 1 to 3 on the basis of softness felt by pressing the surface of the fresh yeast formed body with the finger after thawing.

(Evaluation Criteria of Texture of Formed Body)

Good: The fresh yeast formed body after thawing was as hard as the unfrozen fresh yeast formed body.

Not so good: The fresh yeast formed body after thawing was slightly softer than the unfrozen fresh yeast formed body.

Poor: The fresh yeast formed body after thawing was much softer than the unfrozen fresh yeast formed body.

The thus obtained results are shown in Table 6.

TABLE 6

| | | | | Number | | | |
|---|---|---|---|---|---|---|---|
| | | | | of | Amount | Shape | |
| | | | | freezing | of | retention | Texture |
| | | | | and | generated | ability of | of |
| | | | | thawing | gas | formed | formed |
| | | Shape | Size | cycles | (mL/2h) | body | body |
| Comparative | Unfrozen fresh yeast | Spherical | Diameter 6 cm | — | 206 | Good | Good |
| Example 1 | | | | | | | |
| Example 2 | Frozen fresh yeast | Spherical | Diameter 6 cm | Once | 206 | Good | Good |
| | | | | Twice | 198 | Good | Not so good |
| | | | | Three times | 189 | Not so good | Not so good |
| | | | | Four times | 175 | Poor | Poor |

As can be seen from Table 6, when the frozen fresh yeast formed body according to the present invention was thawed only once, the leavening power was comparable to that of the unfrozen fresh yeast formed body, and the shape and hardness were the same as those of the unfrozen fresh yeast formed body, but when the frozen fresh yeast formed body according to the present invention was subjected to two or more freezing and thawing cycles, the leavening power was reduced, the shape was changed, and softening occurred. From the above results, when the frozen fresh yeast formed body is thawed once, it is better not to freeze it again.

### Test Example 5

The frozen fresh yeast formed body of Example 2 and the unfrozen fresh yeast formed body of Comparative Example 1 were allowed to stand in a refrigerator at 4° C. for 2 days or 1 month and were evaluated for their leavening power in the same manner as in Test Example 4.

The thus obtained results are shown in Table 7.

TABLE 7

| | | | | Amount of generated gas (mL/2 h) | |
|---|---|---|---|---|---|
| | | Shape | Size | After refrigeration for 2 days | After refrigeration for 1 month |
| Comparative | Unfrozen fresh yeast | Spherical | Diameter 6 cm | 206 | 159 |
| Example 1 | | | | | |
| Example 2 | Frozen fresh yeast | Spherical | Diameter 6 cm | 206 | 135 |

As can be seen from Table 7, the leavening power of the frozen fresh yeast formed body and the leavening power of the unfrozen fresh yeast formed body were both significantly reduced after storage under refrigeration for 1 month, but the degree of reduction in the leavening power of the frozen fresh yeast formed body was greater than that of the unfrozen fresh yeast formed body. From the fact, when the frozen fresh yeast formed body is thawed once, it is better to immediately use it without being stored under refrigeration for a long period of time.

### Examples 7 to 12, Comparative Examples 8 to 14, Reference Example 2

Formed bodies and packages containing the formed bodies were obtained in the same manner as in Examples 1 to 6, Comparative Examples 1 to 7, and Reference Example 1 except that KGLY59 (accession number: FERM BP-20635, described in Japanese Patent No. 4839860) was used as a yeast strain. The dry matter weight of yeast in each of the formed bodies was 31.5%. These formed bodies and the packages containing the formed bodies were subjected to various evaluations in accordance with Test Example 1. The thus obtained results are shown in Table 8.

TABLE 8
Forms and evaluation results of fresh yeast formed bodies

| | | | Example | Example | Example | Example | Example | Example | Comparative |
|---|---|---|---|---|---|---|---|---|---|
| | | | 7 | 8 | 9 | 10 | 11 | 12 | Example 8 |
| Form | Shape | | Spherical | Spherical | Spherical | Ellipsoidal | Ellipsoidal | Cylindrical | Spherical |
| | Size | Diameter (cm) | 4.0 | 6.0 | 12.0 | — | — | 4.0 | 6.0 |
| | | Height (cm) | — | — | — | 1.0 | 4.0 | 8.0 | — |
| | | Long side (cm) | — | — | — | 4.0 | 6.0 | — | — |
| | | Short side (cm) | — | — | — | 2.0 | 4.0 | — | — |
| | Maximum contact | | 0 | 0 | 0 | 0 | 0 | 10.0 | 0 |
| | area ratio (%) | | | | | | | | |
| | Density (g/cm³) | | 1.07 | 1.07 | 1.07 | 1.08 | 1.08 | 1.02 | 1.07 |
| | Weight per one | | 36 | 121 | 968 | 4.5 | 54 | 102 | 121 |
| | formed body (g) | | | | | | | | |
| Formulation | Yeast content * 1 (wt %) | | 31.5 | 31.5 | 31.5 | 31.5 | 31.5 | 31.5 | 31.5 |
| | Moisture content (wt %) | | 68.5 | 68.5 | 68.5 | 68.5 | 68.5 | 68.5 | 68.5 |
| | Emulsifier content (wt %) | | — | — | — | — | — | — | — |
| | Fat and oil content (wt %) | | — | — | — | — | — | — | — |
| | Presence or | | Frozen | Frozen | Frozen | Frozen | Frozen | Frozen | Unfrozen |
| | absence of freezing | | | | | | | | |
| | treatment after forming | | | | | | | | |
| | Long-term storage stability | | 5 | 5 | 5 | 5 | 5 | 5 | 1 |
| | Difficulty of disintegration | | 5 | 5 | 5 | 5 | 5 | 5 | 3 |
| | Difficulty of consolidating | | 5 | 5 | 4 | 3 | 5 | 3 | — |
| | frozen formed bodies | | | | | | | | |
| | Ease of dividing | | 5 | 5 | 4 | 4 | 5 | 4 | — |
| | consolidated mass | | | | | | | | |
| | Ease of counting | | 5 | 5 | 4 | 4 | 5 | 4 | 3 |
| | Coefficient of | | 0.002 | 0.002 | 0.012 | 0.002 | 0.002 | 0.008 | 0.003 |
| | variation for weight | | | | | | | | |
| | | | Comparative | Comparative | Comparative | Comparative | Comparative | Comparative | Reference |
| | | | Example 9 | Example 10 | Example 11 | Example 12 | Example 13 | Example 14 | Example 2 |
| Form | Shape | | Cylindrical | Rectangular | Bulky | Spherical | Rectangular | Rectangular | Spherical |
| | | | | parallelepiped | | | parallelepiped | parallelepiped | |
| | Size | Diameter (cm) | 4.0 | — | — | 0.30 | — | — | 0.30 |
| | | Height (cm) | 8.0 | 6.5 | — | — | 6.5 | 6.5 | — |
| | | Long side (cm) | — | 6.5 | — | — | 11.7 | 6.5 | — |
| | | Short side (cm) | — | 5.9 | — | — | 3.3 | 5.9 | — |

| | | | | | | | | |
|---|---|---|---|---|---|---|---|---|
| | Maximum contact | 10.0 | 16.1 | — | 0 | 28.1 | 16.1 | 0 |
| | area ratio (%) | | | | | | | |
| | Density (g/cm³) | 1.02 | 1.05 | — | 1.02 | 1.05 | 1.05 | 1.02 |
| | Weight per one | 102 | 262 | 0.0001-5.0 | 0.014 | 264 | 262 | 0.014 |
| | formed body (g) | | | | | | | |
| Formulation | Yeast content * 1 (wt %) | 31.5 | 31.5 | 31.5 | 31.5 | 31.5 | 31.5 | 32.0 |
| | Moisture content (wt %) | 68.5 | 68.5 | 68.5 | 68.5 | 68.5 | 68.5 | 57.7 |
| | Emulsifier content (wt %) | — | — | — | — | — | — | 0.3 |
| | Fat and oil content (wt %) | — | — | — | — | — | — | 10.0 |
| | Presence or | Unfrozen | Unfrozen | Frozen | Frozen | Frozen | Frozen | Frozen |
| | absence of freezing | | | | | | | |
| | treatment after forming | | | | | | | |
| | Long-term storage stability | 1 | 1 | 1 | 5 | 5 | 5 | 5 |
| | Difficulty of disintegration | 3 | 2 | — | 5 | 5 | 5 | 4 |
| | Difficulty of consolidating | — | — | 1 | 2 | 1 | 1 | 5 |
| | frozen formed bodies | | | | | | | |
| | Ease of dividing | — | — | 1 | 1 | 1 | 2 | 5 |
| | consolidated mass | | | | | | | |
| | Ease of counting | 3 | 3 | 1 | 2 | 3 | 3 | 2 |
| | Coefficient of | 0.003 | 0.003 | 1.730 | 0.024 | 0.140 | 0.100 | 0.020 |
| | variation for weight | | | | | | | |

* 1 in terms of dry matter weight

### Examples 13 to 18, Comparative Examples 15 to 21, Reference Example 3

Formed bodies and packages containing the formed bodies were obtained in the same manner as in Examples 1 to 6, Comparative Examples 1 to 7, and Reference Example 1 except that KCY1254 (accession number: NITE BP-1396, described in Japanese Patent No. 5677624) was used as a yeast strain. The dry matter weight of yeast in each of the formed bodies was 32.4%. These formed bodies and the packages containing the formed bodies were subjected to various evaluations in accordance with Test Example 1. The thus obtained results are shown in Table 9.

TABLE 9
Forms and evaluation results of fresh yeast formed bodies

| | | | Example | Example | Example | Example | Example | Example | Comparative |
|---|---|---|---|---|---|---|---|---|---|
| | | | 13 | 14 | 15 | 16 | 17 | 18 | Example 15 |
| Form | Shape | | Spherical | Spherical | Spherical | Ellipsoidal | Ellipsoidal | Cylindrical | Spherical |
| | Size | Diameter (cm) | 4.0 | 6.0 | 12.0 | — | — | 4.0 | 6.0 |
| | | Height (cm) | — | — | — | 1.0 | 4.0 | 8.0 | — |
| | | Long side (cm) | — | — | — | 4.0 | 6.0 | — | — |
| | | Short side (cm) | — | — | — | 2.0 | 4.0 | — | — |
| | Maximum contact | | 0 | 0 | 0 | 0 | 0 | 10.0 | 0 |
| | area ratio (%) | | | | | | | | |
| | Density (g/cm³) | | 1.07 | 1.07 | 1.07 | 1.08 | 1.08 | 1.02 | 1.07 |
| | Weight per one | | 36 | 121 | 968 | 4.5 | 54 | 102 | 121 |
| | formed body (g) | | | | | | | | |
| Formulation | Yeast content * 1 (wt %) | | 32.4 | 32.4 | 32.4 | 32.4 | 32.4 | 32.4 | 32.4 |
| | Moisture content (wt %) | | 67.6 | 67.6 | 67.6 | 67.6 | 67.6 | 67.6 | 67.6 |
| | Emulsifier content (wt %) | | — | — | — | — | — | — | — |
| | Fat and oil content (wt %) | | — | — | — | — | — | — | — |
| | Presence or | | Frozen | Frozen | Frozen | Frozen | Frozen | Frozen | Unfrozen |
| | absence of freezing | | | | | | | | |
| | treatment after forming | | | | | | | | |
| | Long-term storage stability | | 5 | 5 | 5 | 5 | 5 | 5 | 1 |
| | Difficulty of disintegration | | 5 | 5 | 5 | 5 | 5 | 5 | 3 |
| | Difficulty of consolidating | | 5 | 5 | 4 | 3 | 5 | 3 | — |
| | frozen formed bodies | | | | | | | | |
| | Ease of dividing | | 5 | 5 | 4 | 4 | 5 | 4 | — |
| | consolidated mass | | | | | | | | |
| | Ease of counting | | 5 | 5 | 4 | 4 | 5 | 4 | 3 |
| | Coefficient of | | 0.002 | 0.002 | 0.012 | 0.002 | 0.002 | 0.008 | 0.003 |
| | variation for weight | | | | | | | | |
| | | | Comparative | Comparative | Comparative | Comparative | Comparative | Comparative | Reference |
| | | | Example 16 | Example 17 | Example 18 | Example 19 | Example 20 | Example 21 | Example 3 |
| Form | Shape | | Cylindrical | Rectangular | Bulky | Spherical | Rectangular | Rectangular | Spherical |
| | | | | parallelepiped | | | parallelepiped | parallelepiped | |
| | Size | Diameter (cm) | 4.0 | — | — | 0.30 | — | — | 0.30 |
| | | Height (cm) | 8.0 | 6.5 | — | — | 6.5 | 6.5 | — |
| | | Long side (cm) | — | 6.5 | — | — | 11.7 | 6.5 | — |
| | | Short side (cm) | — | 5.9 | — | — | 3.3 | 5.9 | — |

| | | | | | | | | |
|---|---|---|---|---|---|---|---|---|
| | Maximum contact | 10.0 | 16.1 | — | 0 | 28.1 | 16.1 | 0 |
| | area ratio (%) | | | | | | | |
| | Density (g/cm³) | 1.02 | 1.05 | — | 1.02 | 1.05 | 1.05 | 1.02 |
| | Weight per one | 102 | 262 | 0.0001-5.0 | 0.014 | 264 | 262 | 0.014 |
| | formed body (g) | | | | | | | |
| Formulation | Yeast content * 1 (wt %) | 32.4 | 32.4 | 32.4 | 32.4 | 32.4 | 32.4 | 32.0 |
| | Moisture content (wt %) | 67.6 | 67.6 | 67.6 | 67.6 | 67.6 | 67.6 | 57.7 |
| | Emulsifier content (wt %) | — | — | — | — | — | — | 0.3 |
| | Fat and oil content (wt %) | — | — | — | — | — | — | 10.0 |
| | Presence or | Unfrozen | Unfrozen | Frozen | Frozen | Frozen | Frozen | Frozen |
| | absence of freezing | | | | | | | |
| | treatment after forming | | | | | | | |
| | Long-term storage stability | 1 | 1 | 1 | 5 | 5 | 5 | 5 |
| | Difficulty of disintegration | 3 | 2 | — | 5 | 5 | 5 | 4 |
| | Difficulty of consolidating | — | — | 1 | 2 | 1 | 1 | 5 |
| | frozen formed bodies | | | | | | | |
| | Ease of dividing | — | — | 1 | 1 | 1 | 2 | 5 |
| | consolidated mass | | | | | | | |
| | Ease of counting | 3 | 3 | 1 | 2 | 3 | 3 | 2 |
| | Coefficient of | 0.003 | 0.003 | 1.730 | 0.024 | 0.140 | 0.100 | 0.020 |
| | variation for weight | | | | | | | |

* 1 in terms of dry matter weight

### Examples 19 to 24, Comparative Examples 22 to 28, Reference Example 4

Formed bodies and packages containing the formed bodies were obtained in the same manner as in Examples 1 to 6, Comparative Examples 1 to 7, and Reference Example 1 except that KCY1217 (accession number: NITE BP-1058, described in Japanese Patent No. 5907161) was used as a yeast strain. The dry matter weight of yeast in each of the formed bodies was 32.9%. These formed bodies and the packages containing the formed bodies were subjected to various evaluations in accordance with Test Example 1. The thus obtained results are shown in Table 10.

TABLE 10
Forms and evaluation results of fresh yeast formed bodies

| | | | Example | Example | Example | Example | Example | Example | Comparative |
|---|---|---|---|---|---|---|---|---|---|
| | | | 19 | 20 | 21 | 22 | 23 | 24 | Example 22 |
| Form | Shape | | Spherical | Spherical | Spherical | Ellipsoidal | Ellipsoidal | Cylindrical | Spherical |
| | Size | Diameter (cm) | 4.0 | 6.0 | 12.0 | — | — | 4.0 | 6.0 |
| | | Height (cm) | — | — | — | 1.0 | 4.0 | 8.0 | — |
| | | Long side (cm) | — | — | — | 4.0 | 6.0 | — | — |
| | | Short side (cm) | — | — | — | 2.0 | 4.0 | — | — |
| | Maximum contact | | 0 | 0 | 0 | 0 | 0 | 10.0 | 0 |
| | area ratio (%) | | | | | | | | |
| | Density (g/cm³) | | 1.07 | 1.07 | 1.07 | 1.08 | 1.08 | 1.02 | 1.07 |
| | Weight per one | | 36 | 121 | 968 | 4.5 | 54 | 102 | 121 |
| | formed body (g) | | | | | | | | |
| Formulation | Yeast content * 1 (wt %) | | 32.9 | 32.9 | 32.9 | 32.9 | 32.9 | 32.9 | 32.9 |
| | Moisture content (wt %) | | 67.1 | 67.1 | 67.1 | 67.1 | 67.1 | 67.1 | 67.1 |
| | Emulsifier content (wt %) | | — | — | — | — | — | — | — |
| | Fat and oil content (wt %) | | — | — | — | — | — | — | — |
| | Presence or | | Frozen | Frozen | Frozen | Frozen | Frozen | Frozen | Unfrozen |
| | absence of freezing | | | | | | | | |
| | treatment after forming | | | | | | | | |
| | Long-term storage stability | | 5 | 5 | 5 | 5 | 5 | 5 | 1 |
| | Difficulty of disintegration | | 5 | 5 | 5 | 5 | 5 | 5 | 3 |
| | Difficulty of consolidating | | 5 | 5 | 4 | 3 | 5 | 3 | — |
| | frozen formed bodies | | | | | | | | |
| | Ease of dividing | | 5 | 5 | 4 | 4 | 5 | 4 | — |
| | consolidated mass | | | | | | | | |
| | Ease of counting | | 5 | 5 | 4 | 4 | 5 | 4 | 3 |
| | Coefficient of | | 0.002 | 0.002 | 0.012 | 0.002 | 0.002 | 0.008 | 0.003 |
| | variation for weight | | | | | | | | |
| | | | Comparative | Comparative | Comparative | Comparative | Comparative | Comparative | Reference |
| | | | Example 23 | Example 24 | Example 25 | Example 26 | Example 27 | Example 28 | Example 4 |
| Form | Shape | | Cylindrical | Rectangular | Bulky | Spherical | Rectangular | Rectangular | Spherical |
| | | | | parallelepiped | | | parallelepiped | parallelepiped | |
| | Size | Diameter (cm) | 4.0 | — | — | 0.30 | — | — | 0.30 |
| | | Height (cm) | 8.0 | 6.5 | — | — | 6.5 | 6.5 | — |
| | | Long side (cm) | — | 6.5 | — | — | 11.7 | 6.5 | — |
| | | Short side (cm) | — | 5.9 | — | — | 3.3 | 5.9 | — |

| | | | | | | | | |
|---|---|---|---|---|---|---|---|---|
| | Maximum contact | 10.0 | 16.1 | — | 0 | 28.1 | 16.1 | 0 |
| | area ratio (%) | | | | | | | |
| | Density (g/cm³) | 1.02 | 1.05 | — | 1.02 | 1.05 | 1.05 | 1.02 |
| | Weight per one | 102 | 262 | 0.0001-5.0 | 0.014 | 264 | 262 | 0.014 |
| | formed body (g) | | | | | | | |
| Formulation | Yeast content * 1 (wt %) | 32.9 | 32.9 | 32.9 | 32.9 | 32.9 | 32.9 | 32.0 |
| | Moisture content (wt %) | 67.1 | 67.1 | 67.1 | 67.1 | 67.1 | 67.1 | 57.7 |
| | Emulsifier content (wt %) | — | — | — | — | — | — | 0.3 |
| | Fat and oil content (wt %) | — | — | — | — | — | — | 10.0 |
| | Presence or | Unfrozen | Unfrozen | Frozen | Frozen | Frozen | Frozen | Frozen |
| | absence of freezing | | | | | | | |
| | treatment after forming | | | | | | | |
| | Long-term storage stability | 1 | 1 | 1 | 5 | 5 | 5 | 5 |
| | Difficulty of disintegration | 3 | 2 | — | 5 | 5 | 5 | 4 |
| | Difficulty of consolidating | — | — | 1 | 2 | 1 | 1 | 5 |
| | frozen formed bodies | | | | | | | |
| | Ease of dividing | — | — | 1 | 1 | 1 | 2 | 5 |
| | consolidated mass | | | | | | | |
| | Ease of counting | 3 | 3 | 1 | 2 | 3 | 3 | 2 |
| | Coefficient of | 0.003 | 0.003 | 1.730 | 0.024 | 0.140 | 0.100 | 0.020 |
| | variation for weight | | | | | | | |

* 1 in terms of dry matter weight

### Examples 25 to 30, Comparative Examples 29 to 35, Reference Example 5

Formed bodies and packages containing the formed bodies were obtained in the same manner as in Examples 1 to 6, Comparative Examples 1 to 7, and Reference Example 1 except that KCY1222 (accession number: NUE BP-1059, described in Japanese Patent No. 5907161) was used as a yeast strain. The dry matter weight of yeast in each of the formed bodies was 32.3%. These formed bodies and the packages containing the formed bodies were subjected to various evaluations in accordance with Test Example 1. The thus obtained results are shown in Table 11.

TABLE 11
Forms and evaluation results of fresh yeast formed bodies

| | | | Example | Example | Example | Example | Example | Example | Comparative |
|---|---|---|---|---|---|---|---|---|---|
| | | | 25 | 16 | 27 | 28 | 29 | 30 | Example 29 |
| Form | Shape | | Spherical | Spherical | Spherical | Ellipsoidal | Ellipsoidal | Cylindrical | Spherical |
| | Size | Diameter (cm) | 4.0 | 6.0 | 12.0 | — | — | 4.0 | 6.0 |
| | | Height (cm) | — | — | — | 1.0 | 4.0 | 8.0 | — |
| | | Long side (cm) | — | — | — | 4.0 | 6.0 | — | — |
| | | Short side (cm) | — | — | — | 2.0 | 4.0 | — | — |
| | Maximum contact | | 0 | 0 | 0 | 0 | 0 | 10.0 | 0 |
| | area ratio (%) | | | | | | | | |
| | Density (g/cm³) | | 1.06 | 1.06 | 1.06 | 1.07 | 1.07 | 1.01 | 1.06 |
| | Weight per one | | 36 | 120 | 959 | 4.5 | 54 | 101 | 120 |
| | formed body (g) | | | | | | | | |
| Formulation | Yeast content * 1 (wt %) | | 32.3 | 32.3 | 32.3 | 32.3 | 32.3 | 32.3 | 32.3 |
| | Moisture content (wt %) | | 67.7 | 67.7 | 67.7 | 67.7 | 67.7 | 67.7 | 67.7 |
| | Emulsifier content (wt %) | | — | — | — | — | — | — | — |
| | Fat and oil content (wt %) | | — | — | — | — | — | — | — |
| | Presence or | | Frozen | Frozen | Frozen | Frozen | Frozen | Frozen | Unfrozen |
| | absence of freezing | | | | | | | | |
| | treatment after forming | | | | | | | | |
| | Long-term storage stability | | 5 | 5 | 5 | 5 | 5 | 5 | 1 |
| | Difficulty of disintegration | | 5 | 5 | 5 | 5 | 5 | 5 | 3 |
| | Difficulty of consolidating | | 5 | 5 | 4 | 3 | 5 | 3 | — |
| | frozen formed bodies | | | | | | | | |
| | Ease of dividing | | 5 | 5 | 4 | 4 | 5 | 4 | — |
| | consolidated mass | | | | | | | | |
| | Ease of counting | | 5 | 5 | 4 | 4 | 5 | 4 | 3 |
| | Coefficient of | | 0.002 | 0.002 | 0.012 | 0.002 | 0.002 | 0.008 | 0.003 |
| | variation for weight | | | | | | | | |
| | | | Comparative | Comparative | Comparative | Comparative | Comparative | Comparative | Reference |
| | | | Example 30 | Example 31 | Example 32 | Example 33 | Example 34 | Example 35 | Example 5 |
| Form | Shape | | Cylindrical | Rectangular | Bulky | Spherical | Rectangular | Rectangular | Spherical |
| | | | | parallelepiped | | | parallelepiped | parallelepiped | |
| | Size | Diameter (cm) | 4.0 | — | — | 0.30 | — | — | 0.30 |
| | | Height (cm) | 8.0 | 6.5 | — | — | 6.5 | 6.5 | — |
| | | Long side (cm) | — | 6.5 | — | — | 11.7 | 6.5 | — |
| | | Short side (cm) | — | 5.9 | — | — | 3.3 | 5.9 | — |

| | | | | | | | | |
|---|---|---|---|---|---|---|---|---|
| | Maximum contact | 10.0 | 16.1 | — | 0 | 28.1 | 16.1 | 0 |
| | area ratio (%) | | | | | | | |
| | Density (g/cm³) | 1.01 | 1.04 | — | 1.01 | 1.04 | 1.04 | 1.01 |
| | Weight per one | 101 | 259 | 0.0001-5.0 | 0.014 | 261 | 259 | 0.014 |
| | formed body (g) | | | | | | | |
| Formulation | Yeast content * 1 (wt %) | 32.3 | 32.3 | 32.3 | 32.3 | 32.3 | 32.3 | 32.0 |
| | Moisture content (wt %) | 67.7 | 67.7 | 67.7 | 67.7 | 67.7 | 67.7 | 57.7 |
| | Emulsifier content (wt %) | — | — | — | — | — | — | 0.3 |
| | Fat and oil content (wt %) | — | — | — | — | — | — | 10.0 |
| | Presence or | Unfrozen | Unfrozen | Frozen | Frozen | Frozen | Frozen | Frozen |
| | absence of freezing | | | | | | | |
| | treatment after forming | | | | | | | |
| | Long-term storage stability | 1 | 1 | 1 | 5 | 5 | 5 | 5 |
| | Difficulty of disintegration | 3 | 2 | — | 5 | 5 | 5 | 4 |
| | Difficulty of consolidating | — | — | 1 | 2 | 1 | 1 | 5 |
| | frozen formed bodies | | | | | | | |
| | Ease of dividing | — | — | 1 | 1 | 1 | 2 | 5 |
| | consolidated mass | | | | | | | |
| | Ease of counting | 3 | 3 | 1 | 2 | 3 | 3 | 2 |
| | Coefficient of | 0.003 | 0.003 | 1.730 | 0.024 | 0.140 | 0.100 | 0.020 |
| | variation for weight | | | | | | | |

* 1 in terms of dry matter weight

The results of Examples and Comparative Examples in Tables 8 to 11 using different strains showed the same tendency as those of Examples and Comparative Examples in Table 1. That is, in the case of Examples, the amount of generated gas was kept large even after long-term storage, the frozen formed body was hard to disintegrate, the frozen formed bodies were hard to consolidate together, the frozen formed bodies were easily separated from each other even when consolidated together, and the number of the formed bodies was easily counted. On the other hand, in the case of Comparative Examples 8 to 10, 15 to 17, 22 to 24, and 29 to 31 in which the formed bodies were not frozen, the amount of generated gas was significantly reduced after long-term storage, and the formed body was relatively easily disintegrated. In the case of Comparative Examples 11, 18, 25, and 32 in which bulky formed bodies were obtained, long-term storage ability was poor, the frozen formed bodies were easily consolidated together to form a consolidated mass not easily divided, and the number of the frozen formed bodies was difficult to count. In the case of Comparative Examples 12, 19, 26, and 33 in which formed bodies having a small maximum width were obtained and Comparative Examples 13, 14, 20, 21, 27, 28, 34, and 35 in which rectangular parallelepiped formed bodies were obtained, the frozen formed bodies were easily consolidated together to form a consolidated mass not easily divided, and the number of the frozen formed bodies was difficult to count. In the case of Reference Examples in which additives such as edible fat and oil and an emulsifier were added, the frozen formed bodies were very small, and therefore the number of the frozen formed bodies was difficult to count.

1: A frozen fresh yeast formed body, obtained by freezing fresh yeast,
wherein the frozen fresh yeast formed body has an almost spherical shape having a maximum width of 2 to 20 cm, and
the frozen fresh yeast formed body has a yeast content of 25 to 40% by weight in terms of dry weight and a moisture content of 60 to 75% by weight, with respect to a total weight of the frozen fresh yeast formed body.

2: The frozen fresh yeast formed body according to claim 1, wherein the frozen fresh yeast formed body has the almost spherical shape such that, in a plurality of the frozen fresh yeast formed bodies, a maximum area of contact between the frozen fresh yeast formed bodies is 20% or less of a total surface area of a single frozen fresh yeast formed body.

3: The frozen fresh yeast formed body according to claim 1, which is obtained by freezing fresh yeast having a density of 0.90 to 1.20 g/cm³.

4: A package, comprising:
a container; and
a plurality of the frozen fresh yeast formed bodies according to claim 1 packed in the container.

5: The package according to claim 4, wherein a coefficient of variation for formed body weight among the frozen fresh yeast formed bodies is 0.050 or less.

6: A method for producing a frozen fresh yeast formed body, comprising:
forming fresh yeast into an almost spherical shape having a maximum width of 2 to 20 cm to obtain a formed body; and
freezing the formed body to obtain a frozen fresh yeast formed body,
wherein the frozen fresh yeast formed body has a yeast content of 25 to 40% by weight in terms of dry weight and a moisture content of 60 to 75% by weight, with respect to a total weight of the frozen fresh yeast formed body.

7: A method for producing bread dough, comprising:
kneading the frozen fresh yeast formed body according to claim 1 and other bread dough raw materials to produce bread dough.

8: The method according to claim 7, wherein the frozen fresh yeast formed body is added to and kneaded with at least one of the other bread dough raw materials while the frozen fresh yeast body is in a frozen state without being thawed.

9: A method for making bread, comprising:
cooking bread dough obtained by the method according to claim 7 to obtain bread.

10: A method for preventing consolidation of frozen fresh yeast formed bodies, comprising:
forming fresh yeast into an almost spherical shape having a maximum width of 2 to 20 cm to obtain a formed body; and
freezing the formed body to obtain a frozen fresh yeast formed body,
wherein the frozen fresh yeast formed body has a yeast content of 25 to 40% by weight in terms of dry weight and a moisture content of 60 to 75% by weight, with respect to a total weight of the frozen fresh yeast formed body.

11: The method according to claim 7, wherein the frozen fresh yeast formed body is thawed and then added to and kneaded with at least one of the other bread dough raw materials.

12: The frozen fresh yeast formed body according to claim 1, having the yeast content of 30 to 35% by weight in terms of dry weight and the moisture content of 65 to 70% by weight, with respect to the total weight of the frozen fresh yeast formed body.

13: The frozen fresh yeast formed body according to claim 1, wherein the almost spherical shape is a spherical shape or an ellipsoidal shape.

14: The frozen fresh yeast formed body according to claim 1, wherein the almost spherical shape has the maximum width of 3 to 15 cm.

15: The frozen fresh yeast formed body according to claim 1, wherein the almost spherical shape has the maximum width of 4 to 10 cm.

16: The frozen fresh yeast formed body according to claim 1, wherein the almost spherical shape has a minimum width of 0.5 cm or more.

17: The frozen fresh yeast formed body according to claim 1, having a weight of 10 to 300 g.

18: The frozen fresh yeast formed body according to claim 1, which does not comprise edible fat, edible oil, and an emulsifier.
